(19) United States
(12) Patent Application Publication
(10) Pub. No.: US 2022/0334135 A1
(43) Pub. Date: Oct. 20, 2022

(54) METHODS OF MEASURING COPPER CONCENTRATION IN BIOLOGICAL SAMPLES

(71) Applicant: Alexion Pharmaceuticals, Inc., Boston, MA (US)

(72) Inventors: Mark Ma, Madison, CT (US); Tao Liang, Stamford, CT (US); Ryan Pelto, Middletown, CT (US)

(73) Assignee: Alexion Pharmaceuticals, Inc., Boston, MA (US)

(21) Appl. No.: 17/641,907

(22) PCT Filed: Sep. 11, 2020
(86) PCT No.: PCT/US2020/050368

§ 371 Date: Mar. 10, 2022

Related U.S. Application Data
(60) Provisional application No. 62/899,498, filed on Sep. 12, 2019.
(60) Provisional application No. 62/944,498, filed on Dec. 6, 2019.
(60) Provisional application No. 62/958,432, filed on Jan. 8, 2020.

(51) Int. Cl. G01N 33/84; C07K 16/38; G01N 33/543; G01N 33/68

(52) U.S. Cl. CPC G01N 33/84; C07K 16/38; G01N 33/54326; G01N 33/6848

(57) ABSTRACT

This disclosure relates to methods of measuring copper concentrations in biological samples. More particularly, this disclosure relates to methods of measuring non-ceruloplasmin-bound copper concentrations and/or labile-bound copper concentrations in biological samples. Such methods are particularly useful in management and treatment of metabolism-associated diseases or disorders.

### CROSS-REFERENCE TO RELATED APPLICATIONS

This application claims priority to U.S. application No. 62/899,498, filed Sep. 12, 2019, U.S. application No. 62/944,493, filed Dec. 6, 2019, and U.S. application No. 62/958,432, filed Jan. 8, 2020, the disclosures of which are explicitly incorporated by reference herein.

### BACKGROUND OF DISCLOSURE

### Field of Disclosure

This disclosure relates to methods of measuring copper concentrations in biological samples. More particularly, this disclosure relates to methods of measuring free copper (e.g., non-ceruloplasmin-bound copper concentrations, labile bound copper concentrations, or both) in biological samples. Such methods are particularly useful in management and treatment of metabolism associated diseases or disorders, including, but not limited to, Wilson disease.

### Background

Copper (Cu) is an essential element that plays a critical role in the biochemistry of all organisms and is primarily involved in transfer of electrons by specific cuproenzymes in critical metabolic pathways. The reactivity of copper also contributes to copper toxicity, and thus, copper transport and cellular compartmentalization is specifically regulated. One such regulator is ceruloplasmin (CP), which is a copper-containing plasma ferroxidase that plays an essential role in mammalian iron homeostasis. This protein is a member of the multicopper oxidase family of enzymes, utilizing the electron chemistry of bound copper ions. Carrying more than 90% of plasma copper, it allows delivery of plasma copper to peripheral tissues and excretion of copper into the bile. In general, serum ceruloplasmin level is between 200 μg/mL and 400 μg/mL in normal adults. A serum ceruloplasmin level less than the lower reference limit, 200 μg/mL, is traditionally considered to be diagnostic for Wilson disease (WD).

Wilson disease (also called hepatolenticular insufficiency) is an inherited disease of copper transport. Wilson disease is caused by a variety of genetic mutations in the Cu-loading enzyme ATP7B (in humans). ATP7B facilitates the transfer of Cu to CP and Cu-excretion via biliary canaliculi. The resulting defect in the hepatic excretory pathway leads to accumulation of copper in tissues such as the liver, kidneys, the central nervous system/brain, and the cornea, and copper levels remain elevated without treatment. Specifically, copper accumulation exceeds the capacity of CP, giving rise to free, non-ceruloplasmin bound copper (“NCC”) circulating in the blood and accumulating in tissues and organs. This NCC may loosely bind with plasma proteins (such as, for example, albumin, transcuprein, and low molecular weight peptides or amino acids) to form complexes (“labile-bound copper” or “LBC”). NCC and LBC comprise “free copper”, which may contribute to, and be indicative of, copper toxicities observed in Wilson disease.

Thus, a tool for managing and treating patients with copper metabolism-associated disorders, such as Wilson disease, is the measurement of free copper levels. NCC is a biomarker and recognized endpoint for evaluating copper control according to the American Association for the Study of Liver Diseases (AASLD) and the European Wilson Disease practice guidelines. Under these historically recognized treatment guidelines, however, NCC was not directly measured. Rather, only total blood copper and CP levels were measured directly, and NCC was then estimated from the following calculation:

$$NCC [ µM ] = \frac{\begin{matrix}Total plasma Cu [ µg / L ] - \\ ( 3.15 * ceruloplasmin [ \frac{mg}{L} ] )\end{matrix}}{63.5 [ µg / µmol ]}$$

The calculation is premised on the assumption that six copper atoms are always bound to a single CP molecule, and that NCC and ceruloplasmin concentrations are directly correlated. In reality, CP may show considerable heterogeneity in the number of copper atoms associated per CP molecule. This formula to calculate NCC assumes that six copper atoms bind per one CP, but the copper/CP ratio varies with disease state. In fact, 6-8 copper atoms can actually bind to CP, and in Wilson disease usually fewer than six copper atoms are associated per CP molecule.

As a result, this estimation method of determining NCC has been identified as problematic in the clinical setting and criticized for its inherent shortcomings. For example, use of this method and formula can generate physiologically and numerically impossible negative NCC results. But even though a negative value for NCC is meaningless, negative values were calculated and reported in 20-50% of patients evaluated with this method.

Thus, there remains a need for an efficient and accurate method to determine the concentrations of free copper in biological samples.

In particular, there remains a need for a method to directly measure concentrations of non-ceruloplasmin bound copper (NCC), ceruloplasmin-bound copper (CP-Cu, also referred to as CPC), and labile-bound copper (LBC) in patients with copper metabolism-associated diseases and disorders, including but not limited to Wilson disease.

### SUMMARY OF THE DISCLOSURE

The disclosure provides efficient and accurate methods of measuring copper concentrations in biological samples. Thus, one aspect of the disclosure provides methods of measuring labile-bound copper in a biological sample. Such methods comprise:
removing ceruloplasmin from a biological sample to obtain a non-ceruloplasmin sample;
contacting the non-ceruloplasmin sample with a chelator which binds to labile-bound copper;
removing non-labile-bound copper to obtain a labile-bound copper sample; and
measuring the copper concentration in the labile-bound copper sample.

In at least one embodiment, the methods of measuring labile-bound copper in a biological sample comprise:
contacting a biological sample with an immuno-capture reagent which binds to ceruloplasmin,
removing the captured ceruloplasmin to obtain a non-ceruloplasmin sample;
contacting the non-ceruloplasmin sample with a chelator which binds to labile-bound copper;
removing non-labile-bound copper to obtain a labile-bound copper sample; and
measuring the copper concentration in the labile-bound copper sample.

A further aspect of the disclosures provides kits for measuring copper concentration in a biological sample. In certain embodiments of this aspect, the kit comprises an immuno-capture reagent and instructions for use. In certain other embodiments of this aspect, the kit comprises an immuno-capture reagent, a chelator, and instructions for use.

### BRIEF DESCRIPTION OF THE DRAWINGS

The accompanying drawings are included to provide a further understanding of the methods and materials of the disclosure and are incorporated in and constitute a part of this specification. The drawings illustrate one or more embodiment(s) of the disclosure and, together with the description, serve to explain the principles and operation of the disclosure.

FIG. 1 illustrates different measures of copper in a biological sample, such as blood, serum, or plasma. “Total Copper” is all copper in the biological sample. “Total Mo” is total molybdenum, and generally may serve as a surrogate for estimating the concentration of Mo-containing drugs (such as BC-TTM) due to difficulties in measuring the Mo-containing drugs directly. “CP-Cu” (also known as “CPC”) is copper-ceruloplasmin complex. “ALB-Cu” (also known as “Albumin-Cu”) is copper-albumin complex. “PUF-Cu” (also known as “Pep-Cu”) is plasma ultrafiltration copper and generally is a measure of free copper in extracted samples (containing, for example, low molecular weight peptides to which copper loosely binds) obtained by, for example, ultrafiltration.

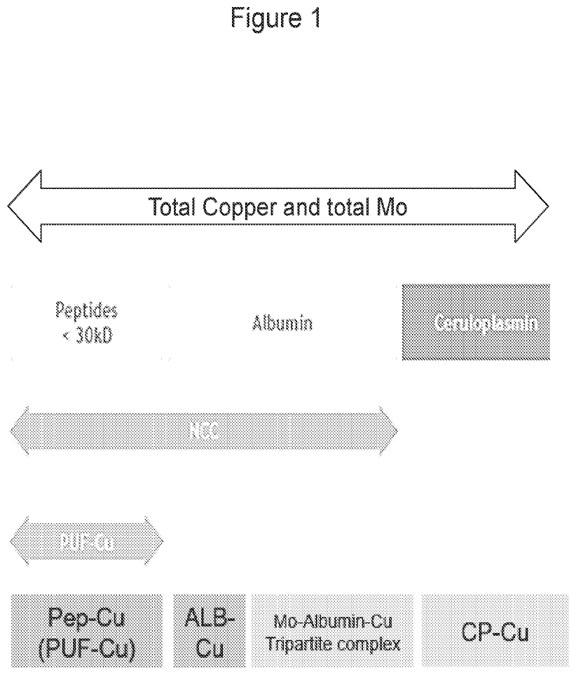

FIG. 2 illustrates a direct measurement of copper in a biological sample according to at least one embodiment of the disclosure.

FIG. 3A illustrates a direct measurement of copper in a biological sample according to at least one embodiment of the disclosure. “Pep-CU” (also known as “PUF-Cu”) is free copper which loosely binds to low molecular weight peptides or amino acids. “MAC” is Mo-albumin tripartite-bound copper.

FIG. 3B illustrates a direct measurement of copper in a biological sample according to at least one embodiment of the disclosure. “UF” is ultrafiltration to remove Pep-Cu. “IP” is immunoprecipitation to remove Pep-Cu.

FIG. 3C illustrates a direct measurement of labile bound copper (comprising Pep-Cu and ALB-Cu) in a biological sample according to at least one embodiment of the disclosure.

FIG. 4 shows the NCC (non-ceruloplasmin-bound copper) measured using the old NCC estimation method (“Old”) and using the instant method (“New”) on Wilson disease baseline (untreated) plasma samples.

FIG. 5 shows the ratio of CPC vs CP for the Wilson disease baseline plasma samples.

### DETAILED DESCRIPTION

Before the disclosed methods and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The term “antibody” as used herein refers to a protein that is capable of recognizing and specifically binding to at least one antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The variable domain may be subjected to further protein engineering to humanize the framework regions if the antibody was derived from a non-human source. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus. Antibody as used herein can include, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. The antibody can also be an engineered protein or antibody-like protein containing at least one immunoglobulin domain (e.g., a fusion protein). The engineered protein or antibody-like protein can also be a bi-specific antibody or a tri-specific antibody, or a dimer, trimer, or multimer antibody, or a diabody, a DVD-Ig, a CODV-Ig, an AFFIBODY® molecule antibody mimetics, or a nanobody.

The term “antibody fragment” or “antigen binding fragment” refers to a portion of an intact or full-length chain or an antibody, generally the target binding or variable region. Examples of antibody fragments include, but are not limited to, F_{ab}, F_{ab′}, F_{(ab′)2} and Fᵥ fragments. As used herein, the term “functional fragment” is generally synonymous with “antibody fragment,” and with respect to antibodies, can refer to antibody fragments such as Fᵥ, F_{ab}, F_{(ab′)2}.

The term “patient” as used herein includes human and animal subjects.

The terms “therapeutic agent” or “therapeutic composition,” as used herein, refer to a compound or composition capable of inducing a desired therapeutic effect when properly administered to a patient.

The term “effective amount” or “therapeutically effective amount” when used in reference to a therapeutic agent or composition refers to an amount or dosage sufficient to produce a desired therapeutic result. More specifically, an effective amount is sufficient to inhibit, for some period of time, one or more of the clinically defined pathological processes associated with the condition being treated. The effective amount may vary depending on the specific therapeutic agent that is being used, and also depends on a variety of factors and conditions related to the patient being treated and the severity of the disorder.

The term “free copper” as used herein refers to free, non-ceruloplasmin-bound copper present in the body of a patient and encompasses NCC and LBC.

### Wilson Disease Symptoms and Treatment

Patients with Wilson disease may experience a combination of hepatic, neurologic, psychiatric, and ophthalmologic symptoms. Hepatic symptoms, for example, may initially present in patients between 9 to 13 years of age and may include both acute liver failure and chronic liver disease. Neurologic signs and symptoms may include, for example, dysarthria, dystonia, tremor, and ataxia. Psychiatric symptoms may range, for example, from irritability to personality changes and depression. Copper deposits in the cornea, also known as Kayser-Fleisher rings, may also be present. Wilson disease symptoms may also present in other locations such as the patient's skin, joints, and kidneys. In addition, a subpopulation of Wilson disease patients is classified as “presymptomatic.” Presymptomatic patients have the genetic mutation for Wilson disease and accompanying biochemical abnormalities but are otherwise asymptomatic.

Without treatment, Wilson disease is fatal; however, with treatment, and when patients are sufficiently compliant with treatment, life expectancy can be normal.

Treatment for Wilson disease targets removing copper accumulated in body tissues followed by preventing re-accumulation of copper. D-penicillamine and trientine are two chelators which may be used to treat symptomatic Wilson disease. D-penicillamine may be considered a first-line therapy; however, some patients require a switch to trientine after experiencing adverse events. Non-limiting examples of penicillamine include CUPRIMINE® (Valeant Pharmaceuticals, Inc.) and DEPEN® (Mylan Specialty LP). Trientine may also be used as a first-line therapy. Non-limiting examples of trientine include trientine hydrochloride (such as SYPRINE® (Valeant Pharmaceuticals, Inc.)) and trientine tetrahydrochloride (such as CUPRIOR® (gmp-orphan SA)). Once a patient's copper levels are reduced, the goal of treatment becomes prevention of copper re-accumulation and maintenance therapy. Zinc salts (non-limiting examples include GALZIN® (zinc acetate) (Teva Pharmaceuticals) and WILZIN® (zine acetate dihydrate) (Recordati Rare Diseases)) may be used for maintenance treatment and may also be used as a first-line therapy in patients including, for example, asymptomatic patients, to reduce copper absorption. In the past, ammonium tetrathiomolybdate has been studied as a potential treatment option. Bis-choline tetrathiomolybdate (BC-TTM), a copper-protein-binding-agent, may also be used for the treatment of Wilson disease. BC-TTM is capable of rapidly forming copper protein complexes with high specificity, de-toxifying free copper in the liver and blood, and promoting biliary excretion of copper. Finally, patients presenting with acute liver failure, decompensated liver disease, or who are unresponsive to treatment may require a liver transplant.

Patients with Wilson disease may be subject to dietary restrictions intended to limit the patients' consumption of copper and copper accumulation. For example, liver should not be ingested during the decoppering period because it can be very high in copper due to the high mineral content of the animals' diets. Shellfish are intermediately high in copper levels, and drinking water can occasionally contain high copper levels as well. Distilled or demineralized water should be used if a patient's drinking water contains more than 0.1 mg of copper per liter. A patient's diet might need to be adjusted so that no more than one or two milligrams of copper are ingested daily. A copper-restricted diet might exclude chocolate, nuts, shellfish, mushrooms, liver, molasses, broccoli, and cereals and dietary supplements enriched with copper, and might be composed of foods with low copper content.

### Methods of Measuring Free Copper

In view of the present disclosure, the methods described herein can be configured by the person of ordinary skill in the art to meet the desired need. In general, the disclosed methods provide improvements in measurement of copper concentration in biological samples. For example, the disclosed methods provide efficient and accurate measurement of free copper in a sample, and eliminate some of the issues associated with the currently used methods, such as biologically impossible negative values of calculated NCC, which is based on incorrect assumptions from the characteristics of fully-functional, non-Wilson disease, CP values. Certain embodiments of the methods of the disclosure provide accurate and reliable quantitation of free copper because the methods of the disclosure are a direct measurement of free copper (i.e., are not a calculated estimate).

Thus, one aspect of the disclosure provides a method of measuring labile-bound copper concentration in a biological sample. In this method, the sample is contacted with an immuno-capture reagent which binds to ceruloplasmin, removing the immuno-captured ceruloplasmin, to obtain a non-ceruloplasmin sample; contacting the non-ceruloplasmin sample with a chelator that binds to labile-bound copper; and removing non-labile-bound copper to obtain a labile-bound copper sample. The copper concentration is measured in the labile-bound copper sample.

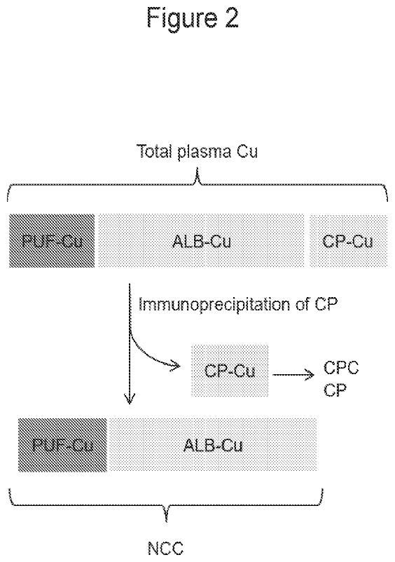

In another aspect of the disclosure, a method of measuring NCC concentration in a biological sample is provided. Such method is schematically shown in FIG. 2. In this method, the sample is contacted with an immuno-capture reagent which binds to ceruloplasmin, removing the captured ceruloplasmin, to obtain a non-ceruloplasmin sample. NCC concentration is measured in the non-ceruloplasmin sample.

In general, any sample containing ceruloplasmin is a biological sample and can be used in the methods of the disclosure. One of the hallmarks of Wilson disease is serum ceruloplasmin concentration of less than 200 μg/mL. Thus, in some embodiments, the biological samples used in the methods of the disclosure are those wherein the ceruloplasmin concentration is less than about 200 μg/mL. In some embodiments, the samples used in the methods of the disclosure are those wherein the ceruloplasmin concentration is in the range of about 200 μg/mL to about 400 μg/mL.

In certain embodiments, the sample is human plasma or human serum. In some embodiments, the sample is human plasma. In some embodiments, the sample is human serum. In some embodiments, the sample is mammalian plasma or mammalian serum.

As noted above, in certain embodiments of the methods of the disclosure as described herein, the sample is contacted with an immuno-capture reagent which binds to ceruloplasmin. Removing the captured ceruloplasmin obtains the non-ceruloplasmin sample. For example, in certain embodiments, the immuno-capture reagent is a ceruloplasmin-capture reagent.

In certain embodiments of the methods of the disclosure as described herein, the immuno-capture reagent is an immunoprecipitating reagent. For example, in certain embodiments, an anti-ceruloplasmin-immobilized solid support can be used as the immunoprecipitating reagent. In some embodiments, the immunoprecipitating reagent is a free anti-ceruloplasmin binding moiety configured to immobilize onto a solid support after complexing with ceruloplasmin. Any suitable solid support known in the art can be used. For example, in certain embodiments, the solid support is at least one solid support selected from magnetic beads, agarose resin, chromatography plate, streptavidin plate, and titer plate. In at least one embodiment, the solid support is magnetic beads. In another embodiment, the solid support is selected from agarose resin, chromatography plate, streptavidin plate, and titer plate.

The solid support can be functionalized with one or more anti-ceruloplasmin reagents selected from a monoclonal antibody, a polyclonal antibody, a chimerized or chimeric antibody, a humanized antibody, a primatized antibody, a deimmunized antibody, a fully human antibody, a bispecific antibody, a diabody, an antigen binding fragment thereof, and a peptide. In certain embodiments of the methods of the disclosure as described herein, the immuno-capture reagent is at least one of a monoclonal or polyclonal goat anti-human ceruloplasmin antibody.

The specific anti-ceruloplasmin reagent, such as the anti-ceruloplasmin antibody, may be selected by evaluating the efficiency of the reagent to bind to CP. For example, the anti-ceruloplasmin reagent may be selected based on its efficiency to deplete CP from a biological sample. In certain embodiments, the anti-CP antibodies showing high efficiency of CP depletion by measuring ceruloplasmin in biological samples post-immuno-capture may be used in the methods of the disclosure. In at least one example embodiment, the antibody with the MS parameters provided in Table 1 may be classified as having high efficiency of the CP depletion (e.g., high CP binding). Thus, in certain embodiments, the anti-ceruloplasmin reagent (e.g., the anti-ceruloplasmin antibody) is capable of depleting at least 90% of CP, e.g., at least 92% or at least 94% of CP, from the total CP in a biological sample. In certain embodiments, the anti-ceruloplasmin reagent is capable of depleting at least 95% of CP, e.g., at least 96% or at least 97% of CP, from the total CP in a biological sample. In certain embodiments, the anti-ceruloplasmin reagent is capable of depleting at least 98% of CP, e.g., at least 98.5%, or at least 99%, or even more than 99% of CP, from the total CP in a biological sample.

TABLE 1

| | Analyte | Internal | | |
|---|---|---|---|---|
| | Peak Area | Standard | Avg. Area | CP |
| Sample | (CP) | Peak Area | Ratio | Depletion % |
| Anti-CP antibody* | 781 | 22122 | 0.0355 | 98.7 |
| No Antibody | 260893 | 22565 | 11.6 | 0 |

In general, measuring the copper concentration may be performed using inductively coupled plasma mass spectrometry (ICP-MS). Other analytical methods suitable for measuring copper concentration can be used including, but not limited to, inductively coupled plasma-optical emission spectroscopy (ICP-OES), and Zeeman graphite furnace atomic absorption spectroscopy (GFAAS).

In certain embodiments of the methods of the disclosure as described herein, prior to measuring the copper concentration, an internal standard (IS) is introduced to the labile-bound copper sample or the non-ceruloplasmin sample. In certain embodiments, the internal standard comprises at least one of copper, rhodium, and indium. In certain embodiments, the internal standard comprises at least one of copper and rhodium.

One of the therapeutic agents used to treat Wilson disease is bis-choline tetrathiomolybdate (BC-TTM), which acts by removing serum copper by associating it with the tetrathiomolybdate anion. When tetrathiomolybdate binds to copper which is associated with proteins in tissue or blood, a tightly bound tripartite complex with the protein/copper (typically albumin/copper) is formed. Formation of this tetrathiomolybdate-copper-albumin tripartite complex is a hallmark of the BC-TTM mechanism of action, and differentiates BC-TTM from chelators, which do not form a protein complex with copper. As a result, molybdenum (Mo) has been used as a surrogate measurement to estimate BC-TTM exposure and adjust effective therapeutic doses. To evaluate NCC in patients treated with BC-TTM, a concept of NCC_{corrected} was implemented in BC-TTM studies, which uses plasma Mo as a measure of tetrathiomolybdate-copper-albumin tripartite complex. NCC_{corrected} is calculated as follows: NCC_{corrected}=(\sqrt{NCC}−0.993\sqrt{totalMo})², where NCC is the estimated NCC calculated according to current procedures.

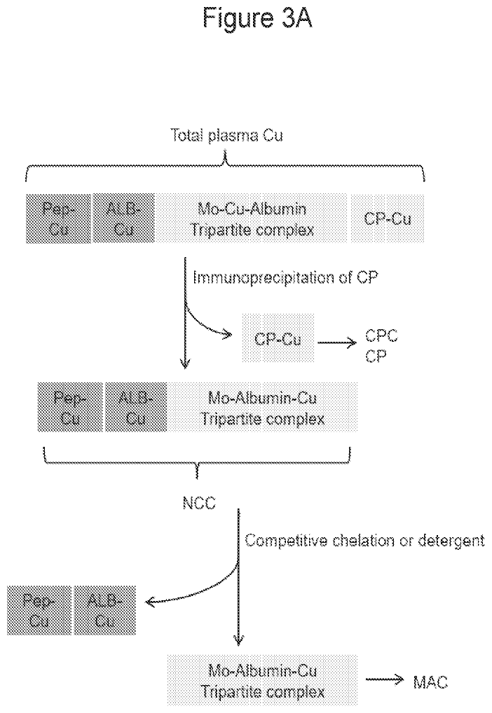

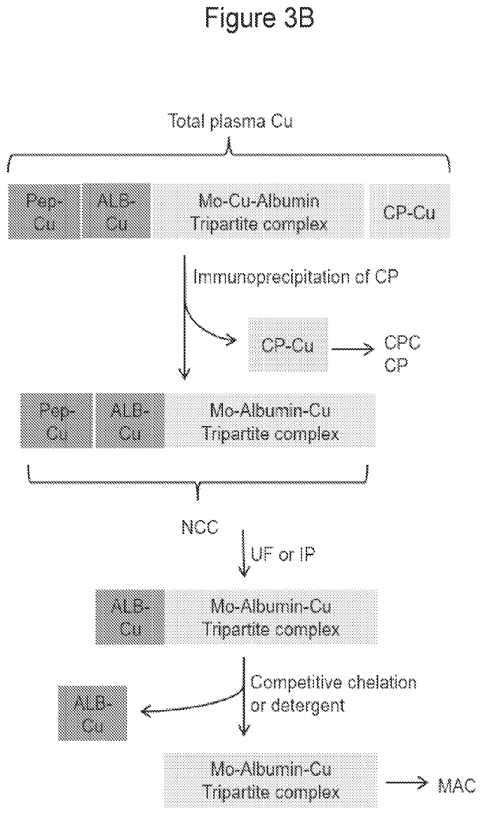

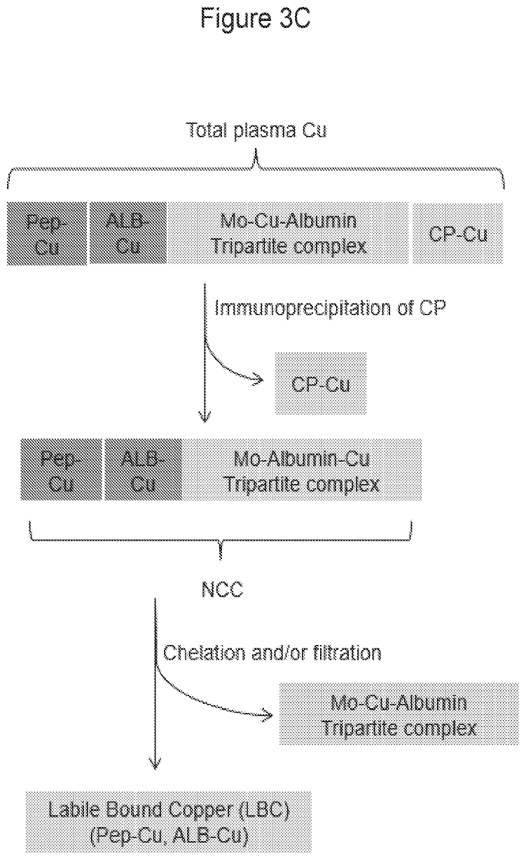

The methods of the disclosure further allow for direct quantification of NCC even in patients receiving BC-TTM. For example, the methods of the disclosure also allow for direct measurement of copper concentration in tetrathiomolybdate-copper-albumin tripartite complex (MAC or Mo-Alb-Cu). Such embodiments are schematically shown in FIGS. 3A and 3B.

Thus, in certain embodiments, the methods of the disclosure as described herein further comprise contacting the non-ceruloplasmin sample with a molybdenum-capture reagent to obtain a molybdenum sample. The molybdenum-capture reagent may be a chelation competition reagent or a detergent. In certain embodiments, the method further comprises measuring a molybdenum-bound copper concentration in the molybdenum sample. The copper concentration can be measured as provided above with respect to measuring copper in the non-ceruloplasmin sample. For example, the copper concentration of the molybdenum sample is measured using inductively coupled plasma mass spectrometry. The accurate non-ceruloplasmin-bound copper concentration may be obtained by subtracting the copper concentration of the molybdenum sample from the copper concentration in the non-ceruloplasmin sample. In some embodiments, the non-ceruloplasmin sample is subjected to ultrafiltration or contacted by an immuno-capture reagent to remove plasma ultrafiltration copper prior to contacting with the molybdenum-capture reagent.

In the methods of the disclosure as described herein, ceruloplasmin is removed by the immune-capture reagent to obtain a non-ceruloplasmin sample and an immuno-captured ceruloplasmin sample. In certain embodiments, the immuno-captured ceruloplasmin sample can be further evaluated. For example, in certain embodiments, the methods of the disclosure further comprise measuring the ceruloplasmin concentration of the immuno-captured ceruloplasmin sample. In general, the ceruloplasmin concentration is measured using mass spectrometry. Other analytical methods suitable for measuring protein concentration in a sample can also be used. In certain embodiments, the mass spectrometry or other analytical methods have an analyte (i.e., ceruloplasmin) detection limit of at least about 5 μg/mL. Measuring the ceruloplasmin concentration, in certain embodiments, is performed using liquid chromatography mass spectrometry (LC-MS). In other embodiments, the methods of the disclosure further comprise measuring the copper concentration of the immuno-captured ceruloplasmin sample. The copper concentration can be measured as provided above with respect to measuring copper in the non-ceruloplasmin sample. For example, the copper concentration of the immuno-captured ceruloplasmin sample is measured using inductively coupled plasma mass spectrometry.

In certain embodiments, the methods of the disclosure as described herein further comprise contacting the non-ceruloplasmin sample with a chelator which binds to labile-bound copper present in the sample. In at least one embodiment, such chelator does not bind copper present in MAC. MAC can then be removed from the sample, leaving a sample comprising labile-bound copper (“labile-bound copper sample”).

Such embodiments are schematically shown in FIG. 3C. In FIG. 3C, the first row shows copper in its various bound forms present in a biological sample, such as, for example, Pep-Cu, Albumin-Cu, Mo-Alb-Cu tripartrite complex, and CP-Cu. In the second row, immunocapture of CP is performed to separate the CP-Cu fraction from the NCC fraction (comprising Pep-Cu, Albumin-Cu, and Mo-Alb-Cu tripartrite complex). In at least one embodiment, the amount of CP-Cu and/or CP in the CP-Cu fraction may be measured. The NCC fraction, shown in the third row of FIG. 3C, may be subjected to chelation and filtration to separate the Mo-Alb-Cu tripartite complex from the LBC fraction (comprising Pep-Cu and Albumin-Cu). In at least one embodiment, the amount of Mo and/or Cu in the Mo-Alb-Cu fraction may be measured. In at least one embodiment, the amount of Cu in the LBC fraction may be measured in accordance with the methods described herein to obtain the amount of labile-bound copper in the sample.

The chelator used in the methods described herein may be chosen from any chelator which binds to labile-bound copper, such as, as non-limiting examples, trientine hydrochloride, trientine tetrahydrochloride, penicillamine, and ethylenediaminetetraacetic acid (also known as EDTA). In at least one embodiment, the chelator comprises EDTA.

Following the addition of the chelator, the resulting sample optionally may be mixed and/or incubated. The MAC may be removed from the non-ceruloplasmin sample by any suitable technique known to those of ordinary skill in the art including, as a non-limiting example, filtration. In at least one embodiment, the sample is centrifuged following removal of the MAC.

### Biomarker for Copper Metabolism

The present disclosure provides for a biomarker for copper metabolism. Free copper concentration in a biological sample is indicative of the concentration of free copper concentration that may be circulating in a patient's blood and accumulating in the patient's tissues and organs. NCC and/or LBC as measured by the methods described herein therefore comprise biomarkers for a patient's copper metabolism. More particularly, NCC and/or LBC as measured by the methods described herein may be used to diagnose, identify, or monitor a patient having a copper-metabolism-associated disorder or disease described herein. In at least one such embodiment, the copper-metabolism-associated disorder or disease is Wilson disease.

In certain embodiments, the biomarker disclosed herein may be measured using the methods for measuring NCC and/or LBC disclosed herein.

The biomarker disclosed herein may be compared to specific, validated reference ranges for free copper concentrations in patients. In at least one embodiment, the biomarker is compared to a set of specific, validated reference ranges for free copper concentrations in particular patient population sub-groups of interest, such as, for example, ethnicity, age, gender, co-morbidities, and other factors.

### Kits for Measuring Copper Concentration

Another aspect of the disclosure provides kits for measuring copper concentration in a biological sample. More particularly, the disclosure provides for kits for measuring free copper concentration (e.g., NCC and/or LBC) in a biological sample.

In certain embodiments of this aspect, the kit for measuring NCC in a biological sample comprises an immuno-capture reagent as described herein and instructions for use. In at least one embodiment, the instructions for use include specific, validated reference ranges for free copper concentrations. In at least one further embodiment, the instructions for use include specific, validated reference ranges for free copper concentrations in particular population sub-groups of interest, such as, for example, ethnicity, age, gender, co-morbidities, and other factors.

In certain other embodiments of this aspect, the kit for measuring LBC in a biological sample comprises an immuno-capture reagent as described herein, a chelator as described herein, and instructions for use as described herein.

In certain other embodiments of this aspect, the kit for measuring LBC in a biological sample comprises an immuno-capture reagent as described herein and instructions for use as described herein. In such certain other embodiments, for example, the instructions for use comprise instructions to use a chelator as described herein to obtain a labile-bound copper sample.

In certain embodiments, the kits disclosed herein may be used to identify or diagnose a patient with a copper-metabolism-associated disorder or disease. In other certain embodiments, the kits disclosed herein may be used to monitor free copper in a patient over time.

### EXAMPLES

Certain aspects of the disclosure are illustrated further by the following examples, which are not to be construed as limiting the disclosure in scope or spirit to the specific methods and materials described in them.

### Example 1: Direct Measurement of Non-Ceruloplasmin Copper (NCC) Bioanalytical Assay

Non-ceruloplasmin copper concentration in human serum/plasma was determined using Inductively Coupled Plasma Mass Spectrometry (ICP-MS) employing a continuous dynode detector.

114 plasma samples were obtained from Phase 3 clinical trials of patients with Wilson disease prior to any treatment with BC-TTM (pre-dose samples). Plasma samples were also obtained from 120 healthy volunteers (60 less than or equal to 18 years old (“Pediatric”), and 60 at least 19 years old (“Adult”)).

Tosyl-activated magnetic beads were obtained commercially (ThermoFisher, formerly Dynabeads) and coated with commercially available goat anti-human ceruloplasmin polyclonal antibodies from Bethyl Laboratories, Inc. (Montgomery, Tex.). Twenty microliters of the human plasma sample were diluted with 200 μL of the coated magnetic beads suspended in PBST solution (phosphate-buffered saline (PBS)+0.01% Tween-20). The dilution was performed in a round bottomed plate for 90 minutes at room temperature with shaking at 1000 rpm. After the incubation, the magnetic beads were separated from the supernatant by a magnetic stand for 5 minutes at room temperature, and subsequently 200 μL of resulting CP-free supernatant was transferred to 15 mL metal-free plastic centrifuge tubes.

The supernatant was measured for Cu concentration directly as described as follows. The CP/magnetic bead fraction was briefly washed and the CP was quantitatively eluted according to standard procedures to provide a solution of CP and CP-Cu (immune-captured CP sample).

100 μL of standard samples including Copper (Cu-STD) and Ceruloplasmin (CP-STD), quality control samples including Copper (Cu-QC) and Ceruloplasmin (CP-QC), and blank samples were each added into individual 15 mL metal-free plastic centrifuge tubes. To each tube containing the plasma sample, blank, STD, or QC, 20 μL of internal standard including Copper (CU-IS) and Ceruloplasmin (CP-IS) and 900 μL 0.1% nitric acid were added, and the tubes were vortexed for about 5 minutes. The tubes were then centrifuged at 12,000 rpm for 5 min at room temperature, and the supernatant was transferred into metal-free plastic tubes suitable for auto-sampler for ICP-MS analysis on Agilent 7800/7900 or Agilent 8900 or LS-MS analysis.

For copper, data acquisition was carried out using Mass Hunter Software (Mass Hunter 4.2 Workstation Software Version C.01.02). For ceruloplasmin, data acquisition was carried out using Analyst Software Version 1.6.2 or equivalent (Applied Biosystems—MDS SCIEX) and chromatograms were integrated using Analyst. The results for the plasma samples are reported in units of concentration as specified by the appropriate bioanalytical method.

Analysis of the CP-free supernatant provided the non-ceruloplasmin-bound copper concentration (NCC). Analysis of the immune-captured CP samples provided the CP-Cu concentration and the CP concentration.

For the 114 pre-dose samples from the Phase 3 clinical trials, all pre-dose samples gave detectable NCC levels, no samples had negative NCC values, and no samples had NCC values below quantitation limits (BQL). The lowest NCC found was 0.2 μM, and the assay sensitivity was determined to be 0.08 μM. Previous analyses reported 40% of samples with negative NCC results.

For the plasma samples obtained from 120 healthy volunteers (60 less than or equal to 18 years old (“Pediatric”) and 60 at least 19 years old (“Adult”)), the lowest NCC found was 0.2 μM, with an overall range of 0.08 μM to 15.7 μM, from baseline clinical trial samples (i.e., samples from untreated Wilson disease patients), corresponding to 5 ng/mL to 1000 ng/mL.

In order to validate the method accuracy and precision, the NCC method was performed on different days, by different analysts, and with multiple QC samples (n=18) for each concentration level. The method showed both accuracy and precision at all concentrations evaluated.

### Example 2: Direct Measurement of Non-Ceruloplasmin Copper (NCC) Bioanalytical Assay

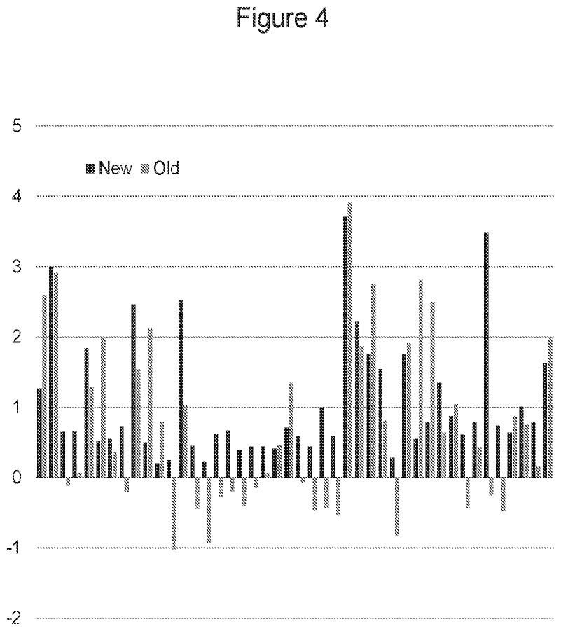

Clinical trial blood samples from (untreated) Wilson disease patients were obtained by sterile venipuncture (or by iv) and stored at 4° C. or on ice until plasma was obtained by centrifuging for 30 min at 2,000 g at 4° C. Plasma samples were stored at −70° C. until analyses were performed. Samples were analyzed according to the process of Example 1. Data is presented in FIG. 4, paired with the historically reported NCC obtained by the prior estimation method. By the old NCC estimation method, approximately 40% of subjects were determined to have negative baseline NCC levels, which would be biologically impossible and which complicates Wilson disease treatment evaluation. In comparison, the method according to Example 1 eliminated negative values of NCC, showing greater sensitivity for quantitating reductions in free Cu.

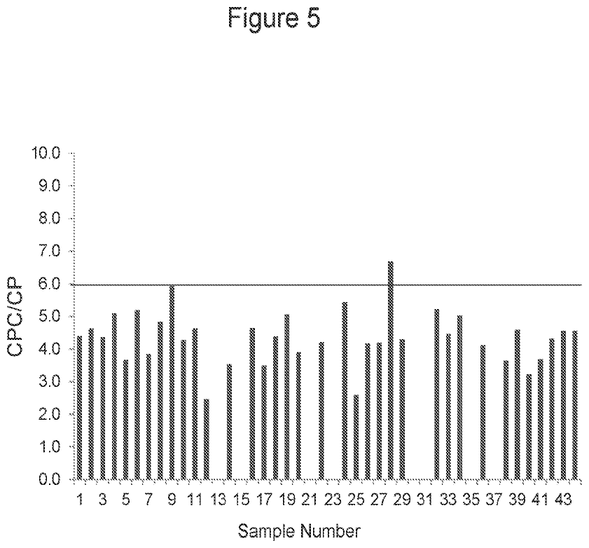

Baseline (untreated) Wilson disease plasma samples (n=42) were also evaluated for the ratio of CP-Cu vs CP to determine the mole ratio of copper to ceruloplasmin protein (FIG. 5). The mean CP-Cu/CP was measured to be 4.29 with a standard deviation of 0.92. This is in contrast to the historically assumed value of 6 Cu per ceruloplasmin. (See Arredondo M. et al., Ceruloplasmin, an indicator of copper status, Biol Trace Elem Res, 2008 Summer; 123(1-3):261-9.)

### Example 3: Labile-Bound Copper (LBC) Bioassay

In this two-step bioassay, the plasma sample was first subject to bead immunocapture of CP, and then was subject to chelation, with subsequent filtration to remove the Mo-Alb-Cu tripartite complex. This method (for the first time) directly measures the free Cu species LBC.

### Preparation of the Immuno-Capture Reagent

Step 1: Preparation of 20 mg/mL Bovine Serum Albumin (BSA) (Trace Copper)

About 20 mg/mL BSA in water was prepared by dissolving about 0.4 g of BSA in about 20 mL of purified H₂O in metal free tubes. About 4000 μL of 20 mg/mL BSA solution were added into a 30K filter (4 mL filter volume) and centrifuged at about 4000× g for about 25 min. The filtrate was discarded. About 3600 μL of 100 mM EDTA solution were added to each filter and the resulting solution was vortexed for about 30 sec and then spun at about 4000× g for about 25 min. The step of discarding the filtrate, adding EDTA, vortexing, and then spinning was repeated another 4 times.

The filtrate was then discarded and about 3600 μL of purified water were added to the filter, and the resulting solution was vortexed for about 30 sec and then centrifuged at about 4000× g for about 15 min. The step of discarding the filtrate, added purified water, vortexing, and then centrifuging was repeated 4 more times. The resulting solution was stored at about −80° C.

Step 2: Preparation of Blocking Buffer: 1×PBS with 0.01% Tween-20

About 0.1 mL of Tween-20 was added to about 1000 mL of 1×PBS buffer. The resulting solution was mixed well and stored at about room temperature.

### Step 3: Preparation of Coupling Buffers

Coupling Buffer A: 0.1 M Borate Buffer, pH 9.5. About 100 mL of 0.5 M borate buffer pH 9.5 was diluted with about 400 mL H₂O. The solution was mixed well and then stored at about 4° C.

Coupling Buffer C: 3M Ammonium Sulfate in Coupling Buffer A. About 39.6 g ammonium sulfate (MW 132.14) was dissolved in about 70-80 mL of Coupling Buffer A. The pH was adjusted to 9.5 using 10M sodium hydroxide solution. An amount of Coupling Buffer A was added to reach final volume of about 100 mL. The solution was mixed well and stored at about 4° C.

Coupling Buffer D (5 mg/mL trace copper BSA in 50 mM tris-HCl, pH 8.5). About 5 mL of 20 mg/mL (trace copper) BSA and 1 mL 1M tris-HCl, pH 8.5 were added to 14 mL of water. The solution was mixed well and stored at about 4° C.

### Step 4: Preparation of Goat Anti-Human Ceruloplasmin-Coated 50 mg Beads

About 30 mg/mL of M-280 tosylactivated magnetic beads (Dynabeads®, from Invitrogen) were mixed well. About 1670 μL (equal to about 50 mg) beads were transferred to a 2 mL Eppendorf tube. The tube was placed on a magnetic stand and about 30 seconds were allowed to pass to allow the beads to settle. The supernatant was removed. The beads were washed with 1.5 mL of Coupling Buffer A with the magnetic stand. The bead suspension was vortexed to ensure fully suspended. About 500 μL Coupling Buffer A and about 600 μL antibody (0.6 mg 1 mg/mL goat anti-human ceruloplasmin antibody (Bethyl)) were added to the beads. Then about 500 μL of Coupling Buffer C was added to the antibody-beads mixture. The mixture was vortexed well. The mixture was incubated overnight (around 18 hours) at 37° C. with rotation. The tube was placed on a magnetic stand and the supernatant removed. About 1 mL of Coupling Buffer D was added and the solution incubated at about 37° C. for about 1 hour with rotation. The tube was placed on a magnetic stand and the supernatant removed. The beads were washed 3 times with about 1.5 mL of Blocking Buffer using the magnetic stand. The beads were resuspended in about 1.25 mL of Blocking Buffer to get a final beads concentration of about 40 mg/mL.

### Plasma Sample Preparation

Human plasma (lithium heparin) samples were obtained from BiolVT. The human plasma samples inherently comprised endogenous LBC, so they were prescreened to determine LBC content. Diluted plasma samples were then prepared using conventional methods.

TABLE 2
Contents of “Diluted LLOQ” and “Diluted Low”
Plasma Samples from Screened Plasma Samples.

| | Amount of | | | | |
|---|---|---|---|---|---|
| | Screened | Amount | Final | | |
| | Plasma | of PBS | Volume | LBC | Mo |
| Name | (μL) | (μL) | (μL) | (ng/mL) | (ng/mL) |
| Diluted LLOQ | 128 | 1872 | 2000 | 5 | N/A |
| Diluted Low | 385 | 1615 | 2000 | 15 | N/A |

TABLE 3
Contents of “Matrix LLOQ QC” and “Matrix Low QC”
Plasma Samples from Diluted Plasma Samples

| | Cu/MO | QC Cu | QC Mo | Dilute with | | | |
|---|---|---|---|---|---|---|---|
| | QC Stock | Stock | Stock | Diluted | Final | Spiked | Spiked |
| | Solution | Solution | Solution | Plasma | Volume | Cu | Mo |
| Name | (ng/mL) | (μL) | (μL) | (μL) | (μL) | (ng/mL) | (ng/mL) |
| Matrix | 200/600 | 25 | 25 | Diluted | 1000 | 5 | 15 |
| LLOQ QC | | | | LLOQ (950) | | | |
| Low | 600/180 | 25 | 25 | Diluted | 1000 | 15 | 45 |
| Matrix QC | | | | Low (950) | | | |

TABLE 4
Contents of “Mid Matrix QC” and “High Matrix QC”
Plasma Samples from Screened Plasma Samples

| | Cu/MO | QC Cu | QC Mo | Dilute with | | | |
|---|---|---|---|---|---|---|---|
| | QC Stock | Stock | Stock | Diluted | Final | | Spiked |
| | Solution | Solution | Solution | Plasma | Volume | Cu | Mo |
| Name | (ng/mL) | (μL) | (μL) | (μL) | (μL) | (ng/mL) | (ng/mL) |
| Mid Matrix | 5000/3000 | 35 | 24 | Plasma | 1000 | 250 | 75 |
| QC | | | | (940) | | | |
| High Matrix | 20000/10000 | 34 | 22 | Plasma | 1000 | 750 | 225 |
| QC | | | | (944) | | | |

### Immunocapture of CP

Each plasma sample to be assayed was thawed, if necessary, to approximately room temperature and then gently vortexed well. About 20 μL of each sample was placed in a well on a 96-well protein low bind plate. About 200 μL of M-280 tosylactivated beads (Dynabeads) pre-coated with anti-human ceruloplasmin antibody (Bethyl Laboratories) in Blocking Buffer prepared as set forth above were added to each well. The plate was sealed and centrifuged at about 500 rpm for approximately 1 minute. The plate was incubated at approximately 25° C. for about 1.5 hours on a plate shaker with speed at about 1000 rpm. The beads were then removed from each well via KingFisher Flex. The solution remaining in each well of the 96-well plate comprised NCC (CP-free) supernatant.

### Chelation-Filtration

About 200 μL of the NCC supernatant for each sample was transferred to a clean, metal-free tube, and then about 60 μL of Chelation Spiking Solution (43.3 mM EDTA and 433.3 μM L-Histidine) were added to each sample. The samples were gently mixed well and then incubated at approximately 37° C. for about 1 hour. Optionally, the tubes could be centrifuged. Each incubated sample was transferred to a washed 30K MWCO centrifugal filter (regenerated cellulose membrane) (Millipore, AmiconUltra) and centrifuged at approximately 14,000×g for about 35 minutes at about 25° C. About 200 μL of filtrate were transferred to a new clean, metal-free plastic tube, and about 600 μL of 0.1% HNO₃ in H₂O were added to the metal-free plastic tubes. About 10 μL of 100 ng/mL rhodium internal standard (“Rhodium IS Spike”) were added to each of the above metal free tubes. Each tube was then centrifuged at approximately 3500 rpm for about 1 minute and vortexed to mix well.

### Preparation of Calibration Standards and Diluted QC Samples in Diluent

Calibration Standard samples and QC samples in Diluent (0.1% HNO₃) for Mass Spectroscopy (MS) analysis as described in Tables 5 and 6 below were prepared using conventional methods in the art.

TABLE 5
Calibration Standards

| | | | Dilute | Cu | Mo |
|---|---|---|---|---|---|
| Label | Amount of Cu | Amount of Mo | with Dil- | (ng/ | (ng/ |
| Name | spiking | spiking | uent to | mL) | mL) |
| STD | 1000 μL of Cu | 600 μL of Mo | 10 mL | 1000 | 300 |
| 8 | Standard Stock 1 | Standard Stock 2 | | | |
| STD | 800 μL of Cu | 480 μL of Mo | 10 mL | 800 | 240 |
| 7 | Standard Stock 1 | Standard Stock 2 | | | |
| STD | 500 μL of Cu | 300 μL of Mo | 10 mL | 500 | 150 |
| 6 | Standard Stock 1 | Standard Stock 2 | | | |
| STD | 300 μL of Cu | 180 μL of Mo | 10 mL | 300 | 90 |
| 5 | Standard Stock 1 | Standard Stock 2 | | | |
| STD | 100 μL of Cu | 60 μL of Mo | 10 mL | 100 | 30 |
| 4 | Standard Stock 1 | Standard Stock 2 | | | |
| STD | 2000 μL of Cu | 1000 μL of Mo | 10 mL | 20 | 6 |
| 3 | Standard Stock 3 | Standard Stock 4 | | | |
| STD | 1000 μL of Cu | 500 μL of Mo | 10 mL | 10 | 3 |
| 2 | Standard Stock 3 | Standard Stock 4 | | | |
| STD | 500 μL of Cu | 250 μL of Mo | 10 mL | 5 | 1.5 |
| 1 | Standard Stock 3 | Standard Stock 4 | | | |

### Cu Standard Stock Solutions:

Cu Standard Stock (1000 μg/mL)
Cu Standard Stock 1 (10 μg/mL)
Cu Standard Stock 2 (1 μg/mL)
Cu Standard Stock 3 (100 ng/mL)

### Mo Standard Stock Solutions

Mo Standard Stock (1001 μg/mL)
Mo Standard Stock 1 (100 μg/mL)
Mo Standard Stock 2 (5 μg/mL)
Mo Standard Stock 3 (1000 ng/mL)
Mo Standard Stock 4 (60 ng/mL)

TABLE 6
QC Samples in Diluent (i.e., 0.1% HNO₃) (no human plasma)

| | Cu Concentration | Mo Concentration |
|---|---|---|
| Label Name | (ng/mL) | (ng/mL) |
| LLOQ | 5 | 1.5 |
| Low QC | 15 | 4.5 |
| Mid QC | 250 | 75 |
| High QC | 750 | 225 |

The Calibration Standard samples were diluted by adding about 100 μL of each Calibration Standard to about 1265 μL of Diluent and mixing well. The QC Samples in Diluent (0.1% HNO₃) were diluted by adding about 100 μL of each QC Sample in Diluent (0.1% HNO₃) to about 1265 μL of Diluent and mixing well. About 200 μL of each of the following samples were placed into a metal free plastic tube and vortexed well: a double blank sample (200 μL 0.1% HNO₃), a blank sample (200 μL 0.1% HNO₃), each of the diluted Calibration Standard samples, and each of the diluted QC Samples in Diluent (0.1% HNO₃). About 600 μL of 0.1% HNO₃ in H₂O (610 μL for double blank) was added to each of the metal free plastic tubes. About 10 μL of Rhodium IS Spike were added to each of the metal free tubes except for the double blank. Each tube was centrifuged at about 3500 rpm for about 1 min. After the centrifuge, each metal free tube was vortexed to mix well.

### Sample Analysis and Results

The samples were analyzed by ICP-MS analysis using an Agilent 8900 with Auto Tune and operating under the conditions and parameters summarized in Tables 7 and 8. A concentric MicroMist nebulizer was used, and the spray chamber temperature was kept at about 2° C. The analysis was performed in He mode.

TABLE 7
Autosampler SPS4

| | | |
|---|---|---|
| Needle rinse | 10 s pump speed 0.3 rps | Purified water |
| Wash 1 | 30 s, pump speed 0.3 rps | 5% HNO₃/H₂O (v/v) |
| Wash 2 | 30 s, pump speed 0.3 rps | 5% HNO₃/H₂O (v/v) |
| Wash 3 | 60 s, pump speed 0.3 rps | 0.1% HNO₃/H₂O (v/v) |

Sample introduction: 30 s, pump speed 0.5 rpm
Stabilize: 35 s, pump speed 0.1 rps

TABLE 8

| | | |
|---|---|---|
| Integration Time/Mass | Cu63 | 1.00 sec |
| | Mo95 | 1.00 sec |
| | Rh ISTD | 0.1 sec |

The LBC bioassay procedure was repeated six times on each type of sample (but only two times for the Calibration Standard samples). The measured LBC concentration and Mo concentration values for the samples are presented in Table 9 below.

TABLE 9
LBC Concentration Results and Mo Concentration Results

| | Cu Concentration | Mo Concentration |
|---|---|---|
| Sample | (ng/mL) | (ng/mL) |
| Diluted LLOQ 6 | 9.4 | 2.8 |
| Diluted LLOQ 5 | 8.9 | 3.0 |
| Diluted LLOQ 4 | 11.2 | 3.1 |
| Diluted LLOQ 3 | 11.8 | 3.0 |
| Diluted LLOQ 2 | 12.1 | 3.0 |
| Diluted LLOQ 1 | 11.8 | 3.0 |
| Diluted Low 6 | 27.6 | 3.0 |
| Diluted Low 5 | 27.6 | 3.5 |
| Diluted Low 4 | 21.7 | 2.9 |
| Diluted Low 3 | 22.7 | 3.1 |
| Diluted Low 2 | 21.0 | 3.1 |
| Diluted Low 1 | 21.9 | 3.0 |
| Matrix LLOQ 6 | 15.0 | 4.8 |
| Matrix LLOQ 5 | 17.9 | 5.0 |
| Matrix LLOQ 4 | 17.9 | 5.0 |
| Matrix LLOQ 3 | 21.0 | 5.0 |
| Matrix LLOQ 2 | 16.9 | 5.7 |
| Matrix LLOQ 1 | 14.5 | 5.3 |
| Low Matrix QC 6 | 36.4 | 8.6 |
| Low Matrix QC 5 | 924.5 | 8.1 |
| Low Matrix QC 4 | 36.9 | 8.3 |
| Low Matrix QC 3 | 35.7 | 8.4 |
| Low Matrix QC 2 | 34.4 | 8.2 |
| Low Matrix QC 1 | 39.4 | 8.5 |
| Mid Matrix QC 6 | 244.9 | 90.0 |
| Mid Matrix QC 5 | 240.7 | 90.0 |
| Mid Matrix QC 4 | 253.3 | 92.6 |
| Mid Matrix QC 3 | 246.1 | 88.7 |
| Mid Matrix QC 2 | 246.4 | 89.6 |
| Mid Matrix QC 1 | 246.3 | 89.4 |
| High Matrix QC 6 | 726.0 | 243.5 |
| High Matrix QC 5 | 749.8 | 252.7 |
| High Matrix QC 4 | 746.0 | 245.0 |
| High Matrix QC 3 | 742.9 | 248.8 |
| High Matrix QC 2 | 733.8 | 249.2 |
| High Matrix QC 1 | 763.4 | 258.4 |
| LLOQ 1 | 5.3 | 1.7 |
| LLOQ 2 | 5.3 | 1.7 |
| LLOQ 3 | 5.2 | 1.7 |
| LLOQ 4 | 6.0 | 1.6 |
| LLOQ 5 | 5.5 | 1.6 |
| LLOQ 6 | 5.5 | 1.7 |
| Low QC 1 | 16.1 | 4.9 |
| Low QC 2 | 16.8 | 4.9 |
| Low QC 3 | 17.1 | 5.1 |
| Low QC 4 | 15.6 | 4.7 |
| Low QC 5 | 16.1 | 4.8 |
| Low QC 6 | 16.1 | 4.8 |
| Mid QC 1 | 244.6 | 69.8 |
| Mid QC 2 | 249.4 | 72.2 |
| Mid QC 3 | 246.8 | 71.8 |
| Mid QC 4 | 252.2 | 70.5 |
| Mid QC 5 | 254.0 | 73.0 |
| Mid QC 6 | 262.1 | 73.8 |
| High QC 1 | 736.9 | 226.8 |
| High QC 2 | 736.4 | 226.6 |
| High QC 3 | 752.2 | 231.7 |
| High QC 4 | 755.4 | 229.7 |
| High QC 5 | 757.5 | 229.7 |
| High QC 6 | 756.3 | 228.3 |
| STD 1 | 5.1 | 1.6 |
| STD 2 | 9.4 | 3.0 |
| STD 3 | 20.1 | 5.9 |
| STD 4 | 99.9 | 29.1 |
| STD 5 | 302.4 | 91.3 |
| STD 6 | 493.4 | 153.9 |
| STD 7 | 803.6 | 241.3 |
| STD 8 | 999.5 | 304.5 |
| STD 1 | 5.1 | 1.5 |
| STD 2 | 9.9 | 2.8 |
| STD 3 | 63.9 | 5.9 |
| STD 4 | 101.0 | 29.4 |
| STD 5 | 308.9 | 91.4 |
| STD 6 | 514.5 | 157.1 |
| STD 7 | 776.6 | 232.4 |
| STD 8 | 991.1 | 303.0 |

### Example 4: Validation of NCC and LBC Bioanalytical Assay

A validation was performed of the NCC bioanalytical assay for measuring non-ceruloplasmin copper (NCC) and ceruloplasmin copper (CPC) in lithium heparin human plasma by ICP-MS. A validation was also performed of the LBC bioanalytical assay for measuring labile-bound copper (LBC) in lithium heparin human plasma by ICP-MS.

### Preparation of Samples

Because copper is an endogenous component in human blood, with total concentrations ranging from approximately 800 to approximately 1200 ng/mL in normal human plasma (versus approximately 20 to 100 ng/mL as non-ceruloplasmin copper), calibration standards in human plasma could not be prepared without significant interference from the endogenous levels of copper. Therefore, 0.1% nitric acid in water (“Diluent”) was used to prepare the calibration standards. For the same reason, the QC samples were also prepared in Diluent. To ensure the accuracy and precision of the assay method including the immunocapture process, however, matrix QC samples were prepared by either diluting plasma (of established copper concentration) with phosphate buffered saline (PBS) or by spiking additional copper in prescreened lithium heparin human plasma with the average endogenous concentration levels taken into consideration to achieve samples having the same Cu levels as the LLOQ QC, Low QC, Mid QC, and High QC samples.

Copper can only be transported onto ceruloplasmin in live subjects with enzymatic assistance; thus, the preparation of validation samples for CPC would be difficult. Only the matrix effect evaluation was conducted for the CPC samples.

Biological Matrix: Blank lithium heparin human plasma, 100% hemolyzed blank lithium heparin human plasma, and blank lithium human whole blood were obtained from BIOIVT.

Blank human plasma was prepared and used during the method validation. The pooled plasma was pre-screened to determine the endogenous concentration level of non-ceruloplasmin copper. The mean endogenous concentrations in the pooled pre-screened plasma were taken into consideration when the matrix QC samples were prepared.

For the hemolysis evaluation, 2% hemolyzed plasma was prepared from 100% hemolyzed lithium heparin human plasma that had been diluted 50-fold with pooled non-hemolyzed plasma. The mean endogenous concentration in the pre-screened 2% hemolyzed plasma was taken into consideration when the Mid and High QC samples were prepared for the hemolysis evaluation. The plasma (pooled and individual lots) was stored at −20° C.±8° C. The whole blood lot was stored at 4° C.±4° C.

Concentrations of Calibration Standards: Calibration standards were prepared in Diluent at the copper concentration levels listed in Table 10. In addition, the calibration standards included a “double blank” (without internal standard) sample in Diluent and a “blank” (with internal standard) sample in Diluent.

TABLE 10
Calibration Standard Cu Concentrations

| Calibration Standard | Copper Concentration, ng/mL |
|---|---|
| STD 1 | 5.00 |
| STD 2 | 10.0 |
| STD 3 | 20.0 |
| STD 4 | 100 |
| STD 5 | 300 |
| STD 6 | 500 |
| STD 7 | 800 |
| STD 8 | 1000 |

Concentrations of QC Samples: QC samples were prepared at the copper concentration levels listed in Table 11. The LLOQ QC, Low QC, Mid 1 QC. Mid 2 QC, and High QC samples were prepared in Diluent (collectively, the “Diluent QC Samples”). The Matrix LLOQ QC, Matrix Low QC, Matrix Mid QC, and Matrix High QC samples (collectively, the “Matrix QC Samples”) were prepared in plasma diluted with phosphate buffered saline (PBS) buffer or by spiking copper in pre-screened lithium heparin human plasma with the average endogenous concentration levels taken into consideration.

Additional Matrix Mid QC and Matrix High QC samples (300 ng/mL and 750 ng/mL) were prepared in pre-screened 2% hemolyzed plasma for the hemolysis effect evaluation. The 2% hemolyzed plasma was prepared from 100% hemolyzed lithium heparin human plasma (blank human plasma that had been diluted 50-fold with pooled non-hemolyzed plasma). The mean endogenous concentration in the pre-screened 2% hemolyzed plasma was taken into consideration when preparing the additional Matrix Mid QC and Matrix High QC samples.

TABLE 11
QC Sample Cu Concentrations

| | | | Cu Concentration, |
|---|---|---|---|
| Quality Control | Matrix | Analyte | ng/mL |
| LLOQ QC | Diluent | Copper | 5.00 |
| Low QC | Diluent | Copper | 15.0 |
| Mid QC | Diluent | Copper | 250 |
| Mid 1 QC | Diluent | Copper | 150 |
| Mid 2 QC | Diluent | Copper | 300 |
| High QC | Diluent | Copper | 750 |
| Matrix LLOQ QC | Diluted pre- | NCC | 5.00 + measured |
| | screened Plasma | | background |
| | | | concentration |
| Matrix Low QC | Diluted pre- | NCC | 15.0 + measured |
| | screened plasma | | background |
| | | | concentration |
| Matrix Mid QC | Pre-screened plasma | NCC | 300 |
| Matrix High QC | Pre-screened plasma | NCC | 750 |
| Matrix LLOQ QC | Diluted pre- | LBC | 5.00 + measured |
| | screened plasma | | background |
| | | | concentration |
| Matrix Low QC | Diluted pre- | LBC | 15.0 + measured |
| | screened plasma | | background |
| | | | concentration |
| Matrix Mid QC | Pre-screened plasma | LBC | 250 |
| Matrix High QC | Pre-screened plasma | LBC | 750 |

Immunocapture of Ceruloplasmin: Goat anti-human ceruloplasmin antibody was immobilized on Dynabeads® using the method set forth in Step 4 of Example 3, except that in the final resuspension step, a BSA buffer (0.5 mg/mL BSA and 0.01% Tween-20 in PBS) was used in place of the Blocking Buffer. Each Matrix QC Sample was subjected to immunocapture of CP according to the “Immunocapture of CP” method set forth in Example 3, except that, in the LBC assay, following the incubation of about 1.5 hours, the sample plates were further centrifuged at about 500 rpm for about 1 minute before using the KingFisher Flex System to remove the beads. The NCC supernatant for each Matrix QC Sample was retained and, in the NCC assay, subjected to NCC quantification as described below.

In the NCC assay, the CP and CPC that immobilized on the coated beads were eluted for CP quantification and CPC quantification. To perform the elution, the removed beads were washed twice with about 300 μL of PBS with 0.01% Tween-20 (“Blocking Buffer”) followed by a wash with about 300 μL of water. The CP on the beads was eluted by about 200 μL of 30 mM HCl for about 10 min.

About 10 μL of GAYPLSIEPIG[(¹³C₅, ¹⁵N)Val]R internal standard spike (2 μg/mL GAYP peptide in water) were added to each sample except the double blank sample, to which about 10 μL of water were added instead. The resulting samples were centrifuged at about 500 rpm for about 1 min and vortexed for about 1 min at low setting. About 50 μL of each sample were transferred to a new well on a new plate for further evaluation, while the remaining solution was stored under about −70° C. for CPC quantification as described below. A dilution factor of about 1.29 could be considered for calculation of CPC.

In the LBC assay, each NCC supernatant retained from the immunocapture step was subjected to chelation and filtration according to the “Chelation-Filtration” method described in Example 3.

NCC, CPC and LBC Quantification: The quantification of NCC, CPC, and LBC was performed via an ICP-MS method using rhodium as the internal standard (or “IS”). In preparation for the quantification, the calibration standards and Diluent QC Samples were diluted with Diluent. About 100 μL of each calibration standard were added to the Diluent and mixed well to make diluted calibration standard samples (dilution can be scaled up or down by a dilution factor of approximately 13.5). The same procedure was repeated with each Diluent QC Sample to make diluted Diluent QC Samples.

Each sample (double blank, blank, the diluted calibration standards, the diluted Diluent QC Samples, the NCC supernatant samples from the immunocapture step, and the eluted CPC solutions from the immunocapture step, except 155 μL were used for the eluted CPC solutions) were pipetted into metal-free plastic tubes and vortexed well.

The Diluent was added to each of the metal-free plastic tubes. About 10 μL of 100 ng/mL rhodium internal standard (“Rhodium IS Spike”) were added to each tube, except for the double blank sample. Each tube was centrifuged at about 3500 rpm for about 1 min.

For the quantification of NCC and CPC, Agilent 7700x ICP-MS autotune and tune check were performed using a tuning solution (Agilent, 28-1GSX2). For the quantification of LBC, Agilent 8900x ICP-MS autotune and tune check were performed using a tuning solution (Agilent, 30-182GSX2). A concentric MicroMist nebulizer was used, and the spray chamber temperature was kept at about 2° C. The analysis was performed in He mode.

### Data Evaluation

The following validation parameters were evaluated: linearity, sensitivity, intra-run and inter-run accuracy and precision, stability, matrix effect (selectivity), hemolysis effect, batch capacity, injection carryover, and chelation robustness.

To perform the evaluation, counts per second (CPS) were determined by MassHunter® Data Acquisition/Processing Software 4.2. Analyte concentrations were obtained from a calibration curve constructed by plotting the CPS versus the concentration using Watson LIMS (Version 7.3).

Concentrations were calculated using linear regression according to the following equation:

y=ax+b

where:
y=CPS ratio of analyte/internal standard
a=slope of the corresponding standard curve
x=concentration of analyte (ng/mL)
b=intercept of the corresponding standard curve

and 1/x² is used as weighting factor.

For calculation of accuracy and precision, the following formulas were used.

$$\% Bias = \frac{Mean measured conc . - Nominal conc .}{Nominal conc .} ⨯ 100 \\ Accuracy : \\ \% CV = \frac{Standard Deviation ( SD )}{Mean measured conc .} ⨯ 100 \\ Precision :$$

Precision and accuracy were reported to one decimal place. All concentration data was reported to three significant figures.

The Linearity of the method was evaluated at a linear range of 5.00-1000 ng/mL for copper. Linear regression (with a weighting factor of 1/x²) was used to produce the best fit for the concentration-detector response relationship for copper in Diluent. All calibration curves met pre-defined acceptance criteria with a coefficient of determination (R²) 0.98.

Sensitivity: The validation was conducted with a target LLOQ of 5.00 ng/mL for copper in Diluent (LLOQ QC) and 5.00 ng/mL+the measured background concentration for copper in human plasma (Matrix LLOQ QC). To evaluate the sensitivity, six QC samples (for each of the Diluent LLOQ QC sample and Matrix LLOQ QC sample) prepared at the concentration level of LLOQ were analyzed as part of the individual accuracy and precision intra-runs and the concentrations calculated with the calibration curve. In addition, in order to calculate the nominal concentration of the Matrix LLOQ QC samples, the diluted matrix used to prepare the Matrix LLOQ QC samples was also analyzed (n=6) in the same batch runs and the mean endogenous concentration determined. The results demonstrated that the method met the pre-defined acceptance criteria for sensitivity in Diluent (accuracy within ±20.0% and % CV no more than 20.0%) and met the pre-defined acceptance criteria for sensitivity in human plasma (accuracy within ±25.0% and % CV no more than 25.0%). Therefore, the method was sensitive enough to determine the analyte at the LLOQ concentration in either Diluent or human plasma.

The Intra-run and Inter-run Accuracy and Precision were validated by analyzing the QC samples at different concentration levels in different runs on different days. Six replicate sample preparations were analyzed at each concentration level. The intra-run and inter-run accuracy and precision of the method were validated at the following QC concentration levels:
Five QC concentration levels prepared in Diluent: 5.00 ng/mL (LLOQ QC), 15.0 ng/mL (Low QC), 250 ng/mL (Mid QC), 150 ng/mL (Mid 1 QC), 300 ng/mL (Mid 2 QC), and 750 ng/mL (High QC).
Four QC concentration levels prepared in screened human plasma: 5.00 ng/mL+mean measured background concentration (Matrix LLOQ QC), 15.0 ng/mL+mean measured background concentration (Matrix Low QC), 250-300 ng/mL (Matrix Mid QC), and 750 ng/mL (Matrix High QC).

The results demonstrated that the intra-run and inter-run accuracy and precision of the method met the pre-defined acceptance criteria in Diluent (% Bias within ±15.0% (within ±20.0% for LLOQ) and % CV no more than 15.0% (20.0% for LLOQ)) and the pre-defined acceptance criteria in human plasma (% Bias within ±20.0% (within ±25.0% for LLOQ) and % CV no more than 20.0% (25.0% for LLOQ)).

The Matrix Effect (Selectivity) is defined as the suppression or enhancement of ionization of analytes by the presence of matrix components in the biological samples. The matrix effect was evaluated for CPC, NCC, and LBC by extracting single replicates of blank human plasma and spiking each lot at the at the Mid QC concentration level (250 ng/mL) and/or High QC concentration level (750 ng/mL for copper) on top of the endogenous concentration level (determined during the same batch run) post-extraction. The matrix factor was calculated according to the following formula:

$$Matrix Factor = \frac{\begin{matrix}Matrix High QC CPS - \\ Endogenous Matrix CPS\end{matrix}}{Neat High QC CPS}$$

The precision (% CV) of the mean of the measured values had to be 20.0%. The results met the pre-defined acceptance criteria.

Hemolysis Effect: To assess the blood hemolysis effect on the quantitation results, Matrix Mid and Matrix High QC samples were prepared in pre-screened 2% hemolyzed plasma. Three replicates of the Matrix Mid and Matrix High QC samples in hemolyzed plasma were extracted and analyzed. The results met the predefined acceptance criteria: mean concentration within ±20.0% of the nominal concentration and the % CV no more than 20.0%.

Bench-top and Short-term Stability: To assess the bench-top and short-term stability, QC samples at multiple concentration levels were initially stored for approximately 5-11 days at −70° C.±10° C. Three aliquots at each level of frozen QC samples were then thawed at ambient room temperature on the bench-top and a separate three aliquots at each level of frozen QC samples were thawed in the refrigerator at 4° C.±4° C. for 17.5-19 hours. Each aliquot was assayed against the calibration standards. Non-ceruloplasmin copper and labile-bound copper was considered stable if the mean of the obtained concentrations at each level was within ±20.0% of the nominal concentrations and the % CV was no more than 20.0%.

The results demonstrated that the NCC QC samples were stable at ambient room temperature and at 4° C.±4° C. for 19 hours. The results further demonstrated that the LBC QC samples were stable at ambient room temperature and at about 4° C. for 17.5 hours.

QC Freeze/Thaw Stability: The stability through four freeze/thaw cycles was assessed at two QC levels (Matrix Mid QC and Matrix High QC, prepared in pre-screened human plasma) at each concentration level. The QC samples were stored at −70° C.±10° C. and were subjected to four freeze/thaw (ambient room temperature) cycles and the concentrations were measured against calibration standards. The samples for the first freeze cycle were stored in the freezer for at least 24 hours prior to being thawed at ambient room temperature for 60 minutes. The subsequent freeze periods were a minimum of 12 hours duration prior to being thawed under the same conditions as the initial cycle. The QC samples were considered stable if the mean of the obtained concentrations at each level was within ±20.0% of the nominal concentrations and the % CV was no more than 20.0%. The cycle 4 results demonstrated QC samples were stable for at least four freeze/thaw cycles.

Whole Blood Stability. The stability of NCC in human whole blood (lithium heparin as the anticoagulant) in an ice-water bath (wet ice) and at ambient room temperature at three time points (0, 60, and 120 minutes) was evaluated at the endogenous level. The blood samples were stored in an ice-water bath and at ambient room temperature for 0, 60, and 120 minutes before plasma was separated by centrifuging at approximately 3500 rpm at about 4° C. for about 15 minutes. The samples (n=3 separate tubes per storage condition/per time point) were then assayed according to the NCC bioanalytical assay method set forth in this Example 4. The mean CPS ratio for the whole blood samples stored in an ice-water bath and at ambient room temperature for up to 120 minutes were within the ±20.0% acceptance criteria. Therefore, it can be concluded that NCC in human whole blood was stable for 120 minutes in an ice-water bath and at ambient room temperature.

The Injection Carry-over Test is used to evaluate the extent of carryover of the analyte of interest from one sample to the next in each analytical run. A duplicate double blank sample (prepared in Diluent) was injected following the high standard from the set of calibrators during each validation run. For the results to be acceptable, the analyte CPS of the carryover sample injected had to be 525% the mean analyte CPS of the LLOQ (standard 1 samples) for NCC and 520% the mean analyte CPS of the LLOQ for LBC. For the same carryover samples the CPS of the IS had to be 510% the mean IS CPS for NCC and ≤5% the mean IS CPS for LBC and from the accepted batch calibration standards and QC samples. All evaluations met the pre-defined acceptance criteria.

Batch Size Robustness: A validation run was used to mimic the batch size in a sample analysis run. A total of 150-199 samples were run in the batch, which included both a standard curve and bracketing QC samples. The Matrix Low, Mid, and High QC samples were prepared in pre-screened human plasma and used to assess the maximum batch size.

To calculate the nominal concentration of the Matrix Low QC samples, which was determined for each injection cycle of n=6 Matrix Low QC samples during this batch run, the diluted matrix used to prepare the Matrix Low QC samples was also analyzed (n=6) and the mean endogenous concentration determined.

The standards and the QC samples met the general batch acceptance criteria for a sample analysis run (the overall accuracy should be within 20% of the nominal value for QC prepared in plasma or diluted plasma; the CV should be no more than 20% for QC prepared in plasma or diluted plasma; and at least 50% of the QC samples should be within 20% of the nominal concentration at each concentration level for QC prepared in plasma or diluted plasma, and no more than ⅓ of all QC samples should be out of specification).

Selectivity was evaluated by spiking Matrix Mid QC level concentration (250 ng/mL) into six individual lots of human plasma with singlet on top of the LBC endogenous level. The six lots of unspiked plasma samples were analyzed in the same batch run with singlet. The formula to calculate LBC recovery of spiked Matrix Mid QC is as follows:

$$Accuracy \% = \frac{observed concentration}{\begin{matrix}spiked Matrix Mid concentration + \\ endogenous concentration\end{matrix}} \%$$

At least five out of six individual plasma lots should have recovery within 100±20% of the nominal concentration. All plasma lots met the acceptance criteria.

The Chelation Robustness Test examined if a longer incubation time during the chelation step would affect the stability of the Mo-Alb-Cu tripartite complex (“TPC”), resulting in the release of copper into the LBC fraction. Six replicates of Matrix Mid and Matrix High QC samples which had been spiked with TPC were evaluated. During sample preparation, the tested samples were kept in the chelation solution for two hours (1-hour incubation time is currently used in the LBC method) prior to the next step. Extracted samples were measured against freshly-prepared calibration standards.

Six replicates of Matrix Mid and Matrix High QC samples (without TPC spiked) were analyzed in the same batch run to assess the impact of the longer chelation step (at least 2 hours but using the same concentration of EDTA) on copper recovery. The results demonstrate that an incubation time of at least 2 hours for the chelation step does not affect the stability of the TPC.

The results obtained from the method validation demonstrated adequate intra-run and inter-run accuracy and precision, sensitivity, linearity, bench-top stability, short-term stability (4° C.±4° C.), matrix effect, maximum batch-size evaluation, freeze/thaw stability, carry-over evaluation, whole blood stability, and analyte interference on IS. The direct NCC and CPC quantification methods were determined to be suitable for the determination of non-ceruloplasmin copper and ceruloplasmin copper in lithium heparin human plasma. The LBC bioassay method was also determined to be suitable for the determination of labile bound copper in lithium heparin human plasma.

### Example 5: Direct Measurement of Non-Ceruloplasmin Copper (NCC) in Plasma Samples from 24 Healthy Volunteers

Plasma samples were obtained from a Phase 1 clinical trial of twenty-four adult healthy volunteers. The samples were assayed according to the NCC bioassay process outlined in Example 4. The NCC concentration results for the 24 adult healthy volunteers are set forth in Table 12 below.

TABLE 12
NCC Concentrations of 24 Healthy Volunteers

| | NCC Concentration |
|---|---|
| Healthy Volunteer | (ng/mL) |
| 1 | 44.9 |
| 2 | 30.5 |
| 3 | 40.7 |
| 4 | 64.9 |
| 5 | 42.7 |
| 6 | 49.6 |
| 7 | 44.9 |
| 8 | 41.3 |
| 9 | 39.9 |
| 10 | 50.6 |
| 11 | 36.5 |
| 12 | 39.2 |
| 13 | 55.3 |
| 14 | 47.6 |
| 15 | 44.9 |
| 16 | 46.4 |
| 17 | 39.2 |
| 18 | 41.2 |
| 19 | 49.7 |
| 20 | 41.5 |
| 21 | 43.7 |
| 22 | 52.3 |
| 23 | 41.3 |
| 24 | 43.2 |

### Example 6: Healthy Cu Plasma Level Full Validation

Non-affected healthy individuals have their blood drawn and tested according to one or more of the previous Examples. The resulting Cu levels are evaluated and subdivided according to ethnicity, age, gender, co-morbidities, and other factors. Reference levels will be determined with standard deviations for each sub-population. A minimum of 120 individuals are evaluated per sub-group.

### Example 7: Kit for Wilson Disease Diagnosis

Phlebotomists provide sterile, non-copper containing blood sampling centrifuge tube (i.e. standard vacutainer) and venipuncture and obtain a sample of blood from an individual. The sample is spun down to separate the plasma, and cell-free plasma is provided to the testing lab which has been provided with a Cu measurement kit. Components of the kit include anti-ceruloplasmin antibody-coated magnetic beads at standard concentration, EDTA standard concentration solution, and instructions for use. Specific validated reference ranges are provided for particular sub-groups of interest.

### Example 8: Treatment of Wilson Disease

Patients presenting with symptoms believed to be copper metabolism-related have blood samples taken and analyzed according to one or more of the previous Examples. Patients with Wilson disease are treated with at least one therapeutic agent selected from at least one of BC-TTM, trientine hydrochloride, trientine tetrahydrochloride, zinc (or salts thereof), and/or penicillamine, and have blood tests performed according to one or more of the preceding Examples over time (every week, every other week, every month, etc.) during their treatment, and their dosages are modulated in order to maintain a therapeutic level of the therapeutic agent and satisfactory Cu/NCC/LBC levels.

Various aspects of the disclosure are further exemplified by the non-limiting embodiments recited in the claims below. In each case, features of multiple claims can be combined in any fashion not inconsistent with the specification and not logically inconsistent.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of measuring labile-bound copper concentration in a biological sample, the method comprising:
contacting the biological sample with an immuno-capture reagent which binds to ceruloplasmin to form immuno-captured ceruloplasmin,
removing the immuno-captured ceruloplasmin to obtain a non-ceruloplasmin sample;
contacting the non-ceruloplasmin sample with a chelator which binds to labile-bound copper;
removing non-labile-bound copper to obtain a labile-bound copper sample; and
measuring copper concentration in the labile-bound copper sample.

2. The method of claim 1, wherein the sample comprises human plasma or human serum.

3. The method of claim 2 or 3, wherein the immuno-capture reagent comprises an immunoprecipitating reagent.

4. The method of any one of claims 1-3, wherein the immuno-capture reagent comprises a ceruloplasmin-capture reagent.

5. The method of claim 4, wherein the ceruloplasmin-capture reagent comprises at least one reagent selected from a monoclonal antibody, a polyclonal antibody, a bispecific antibody, a diabody, a peptide, and an antigen binding fragment thereof.

6. The method of claim 4, wherein the ceruloplasmin-capture reagent comprises at least one of a monoclonal or polyclonal goat anti-human ceruloplasmin antibody.

7. The method of claim 4, wherein the immuno-capture reagent comprises an anti-ceruloplasmin-immobilized solid support.

8. The method of claim 4, wherein the immunoprecipitating reagent comprises a free anti-ceruloplasmin reagent configured to immobilize onto a solid support after complexing with ceruloplasmin.

9. The method of claim 7 or 8, wherein the solid support comprises magnetic beads.

10. The method of claim 7 or 8, wherein the solid support comprises at least one solid support comprising at least one of agarose resin, chromatography plate, streptavidin plate, and titer plate.

11. The method of any one of claims 1-10, wherein the measuring of the copper concentration in the labile-bound copper sample comprises using inductively coupled plasma mass spectrometry (ICP-MS).

12. The method of any one of claims 1-11, further comprising introducing an internal standard to the labile-bound copper sample prior to the measuring of the copper concentration.

13. The method of claim 12, wherein the internal standard comprises at least one of copper and rhodium.

14. The method of any one of claims 1-13, wherein the removing of the immuno-captured ceruloplasmin further comprises obtaining an immuno-captured ceruloplasmin sample.

15. The method of claim 14, further comprising measuring ceruloplasmin concentration in the immuno-captured ceruloplasmin sample.

16. The method of claim 15, wherein the measuring of the ceruloplasmin concentration comprises using mass spectrometry.

17. The method of claim 16, wherein the mass spectrometry has an analyte detection limit of at least about 5 μg/mL.

18. The method of claim 16 or 17, wherein the measuring of the ceruloplasmin concentration comprises using liquid chromatography mass spectrometry (LC-MS).

19. The method of any of claims 15-18, wherein the ceruloplasmin concentration in the biological sample is less than about 200 μg/mL.

20. The method of any one of claims 15-19, further comprising measuring copper concentration in the immuno-captured ceruloplasmin sample.

21. The method of claim 20, wherein the measuring of the copper concentration in the immune-captured ceruloplasmin sample comprises using inductively coupled plasma mass spectrometry.

22. The method of any one of claims 1-21, wherein the chelator comprises at least one of penicillamine, trientine hydrochloride, trientine tetrahydrochloride, and EDTA.

23. The method of claim 22, wherein the chelator comprises EDTA.

24. The method of any one of claim 1-23 wherein the removing of the non-labile-bound copper further comprises obtaining a non-labile-bound copper sample.

25. The method of any one of claim 24, further comprising measuring copper concentration in the non-labile-bound copper sample.

26. The method of any one of claim 24 wherein the non-labile bound copper sample comprises molybdenum.

27. The method of claim 26 further comprising measuring molybdenum concentration in the non-labile bound copper sample.

28. A kit for measuring copper concentration in a biological sample, the kit comprising: an immuno-capture reagent; and instructions for use.

29. The kit of claim 28, wherein the immuno-capture reagent comprises an immunoprecipitating reagent.

30. The kit of any one of claims 28-29, wherein the immuno-capture reagent comprises a ceruloplasmin-capture reagent.

31. The kit of claim 30, wherein the ceruloplasmin-capture reagent comprises at least one reagent selected from a monoclonal antibody, a polyclonal antibody, a bispecific antibody, a diabody, a peptide, and an antigen binding fragment thereof.

32. The kit of claim 30, wherein the ceruloplasmin-capture reagent comprises at least one of a monoclonal or polyclonal goat anti-human ceruloplasmin antibody.

33. The kit of claim 30, wherein the immuno-capture reagent comprises an anti-ceruloplasmin-immobilized solid support.

34. The kit of claim 30, wherein the immunoprecipitating reagent comprises a free anti-ceruloplasmin reagent configured to immobilize onto a solid support after complexing with ceruloplasmin.

35. The kit of claim 33 or 34, wherein the solid support comprises magnetic beads.

36. The kit of claim 33 or 34, wherein the solid support comprises at least one solid support comprising at least one of agarose resin, chromatography plate, streptavidin plate, and titer plate.

37. A kit for measuring copper concentration in a biological sample, the kit comprising: an immuno-capture reagent; a chelator; and instructions for use.

38. The kit of claim 37, wherein the immuno-capture reagent comprises an immunoprecipitating reagent.

39. The kit of any one of claims 37-38, wherein the immuno-capture reagent comprises a ceruloplasmin-capture reagent.

40. The kit of claim 39, wherein the ceruloplasmin-capture reagent comprises at least one reagent selected from a monoclonal antibody, a polyclonal antibody, a bispecific antibody, a diabody, a peptide, and an antigen binding fragment thereof.

41. The kit of claim 39, wherein the ceruloplasmin-capture reagent comprises at least one of a monoclonal or polyclonal goat anti-human ceruloplasmin antibody.

42. The kit of claim 39, wherein the immuno-capture reagent comprises an anti-ceruloplasmin-immobilized solid support.

43. The kit of claim 39, wherein the immunoprecipitating reagent comprises a free anti-ceruloplasmin reagent configured to immobilize onto a solid support after complexing with ceruloplasmin.

44. The kit of claim 42 or 43, wherein the solid support comprises magnetic beads.

45. The kit of claim 42 or 43, wherein the solid support comprises at least one solid support comprising at least one of agarose resin, chromatography plate, streptavidin plate, and titer plate.

46. The kit of any one of claims 37-45, wherein the chelator comprises at least one of penicillamine, trientine hydrochloride, trientine tetrahydrochloride, and EDTA.

47. The kit of any of one of claims 37-45, wherein the chelator comprises EDTA.
